(19) United States
(12) Patent Application Publication
(10) Pub. No.: US 2004/0224340 A1
(43) Pub. Date: Nov. 11, 2004

(54) Displacing a plasmid in a bacterial population

(76) Inventor: Marcin S. Filutowicz, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/828,679

(22) Filed: Apr. 21, 2004

Related U.S. Application Data
(60) Provisional application No. 60/494,973, filed on Aug. 14, 2003.
(60) Provisional application No. 60/464,443, filed on Apr. 21, 2003.

(51) Int. Cl. C12Q 1/68; C12N 1/21

(52) U.S. Cl. 435/6; 435/455; 435/325; 435/252.3; 435/320.1

(57) ABSTRACT

The present invention provides a conjugative displacing plasmid, a donor cell and a method for eliminating an unwanted trait from target bacteria without killing the bacteria wherein the unwanted trait is conferred by a plasmid.

### CROSS-REFERENCE TO RELATED APPLICATION

This application claims the benefit of U.S. provisional patent application Ser. No. 60/494,973, filed on Aug. 14, 2003, and U.S. provisional patent application Ser. No. 60/464,443, filed on Apr. 21, 2003, both of which are incorporated by reference in their entirety.

### STATEMENT REGARDING FEDERALLY SPONSORED RESEARCH OR DEVELOPMENT

This invention was made with United States government support awarded by the following agency: NIH GM40314. The United States has certain rights in this invention.

### BACKGROUND OF THE INVENTION

As the use of conventional pharmaceutical antibiotics (herein referred to as antibiotics) increases for medical, veterinary and agricultural purposes, the increasing emergence of antibiotic-resistant strains of pathogenic bacteria is an unwelcome consequence. This has become of major concern inasmuch as drug resistance of bacterial pathogens is presently the major cause of failure in the treatment of infectious diseases. Indeed, people now die of certain bacterial infections that previously could have been easily treated with existing antibiotics. Such infections include, for instance, Staphylococcus pneumoniae, causing meningitis, Enterobacter sp., causing pneumonia, Enterococcus sp., causing endocarditis, Yersinia pestisis, causing bubonic plague, Bacillus anthracis, causing anthrax, and Mycobacterium tuberculosis, causing tuberculosis.

As another example, streptomycin has been used in the U.S. to prevent fire blight epidemics. After initial success, however, the streptomycin-resistant strains become so predominant in many of the treated orchards that oxytetracycline is being used instead (McManus and Stockwell, 2001). A very significant fraction of the streptomycin-resistance is plasmid-borne. Oxytetracycline, however, could cause even more severe problems since closely related tetracycline not only selects for maintenance of tetracycline-resistance genes acquired by new recipients but also enhances the transfer of these genes (Saylers, 1993). Although streptomycine and oxytetracycline are effective in preventing fire blight, it is evident that antibiotics introduced into orchards drive resistance development directly within food chain, not only streptomycin- and tetracycline-resistance genes, but other genes that are frequently genetically linked on the same plasmid (Clinical Infectious Diseases: “The need to Improve Antimicrobial Use in Agriculture—Ecological and Human Health Consequences” (2002) vol 34 Supplement 3; Levy, 1998).

The emergence of single- or multi-drug resistant bacteria results from a gene mobilization that responds quickly to the strong selective pressure that is a consequence of antibiotic uses. Over the last several decades, the increasingly frequent usage of antibiotics has acted in concert with spontaneous mutations arising in the bacterial gene pool to produce antibiotic resistance in certain strains. This gene pool is continually utilized by previously sensitive strains capable of accessing it by various means including the transfer of plasmids. As a result, single- and multi-drug resistance genes are commonly found in a large variety of bacterial plasmids (Clinical Infectious Diseases: The Need to Improve Antimicrobial Use in Agriculture—Ecological and Human Health Consequences (2002) vol 34 Supplement 3).

Presently, there is no known method for avoiding the selection of antibiotic resistant bacterial mutants that arise as a result of the many standard applications of antibiotics in the modem world. To control the resistant mutants, new antibiotics can be developed. However, this does not solve the problem that resistant strains will evolve and it is uncertain whether new antibiotic development can keep pace with resistance development. Accordingly, a need exists to develop alternative strategies of antibacterial treatment.

Interest in the use of bacteriophages to treat infectious bacterial diseases developed early in the twentieth century and has undergone a resurgence in recent years. For instance, bacteriophages have been shown effective in the treatment of certain pathogenic E. Coli species in laboratory and farm animals, and have been proposed as a viable alternative to the use of antibiotics (Smith & Huggins, J. Gen. Microbiol. 128: 307-318, 1981; Smith & Huggins, J. Gen. Microbiol. 129: 2659-2675, 1983; Smith et al., J. Gen. Microbiol. 133: 1111-1126, 1986; Kuvda et al., Appl. Env. Microbiol. 65: 3767-3773, 1999; W. C. Summers “Bacteriophage Therapy,” Annu. Rev. Microbiol. 55: 437-451, 2001). However, the use of bacteriophages as antimicrobial agents has certain limitations. First, the relationship between a phage and its host bacterial cell is typically very specific, such that a broad host-range phage agent is generally unavailable. Second, the specificity of interaction usually arises at the point of the recognition and binding of the phage to the host cell. This often occurs through the expression of surface receptors on the host cell to which the phage specifically binds. Inasmuch as such receptors are usually encoded by a single gene, mutations in the host bacterial cell to alter the surface receptor, thereby escaping detection by the phage, can occur with a frequency equivalent to or higher than, the mutation rate to acquire antibiotic resistance. As a result, if phage were utilized as commonly as antibiotics, resistance of pathogenic bacteria to phages could become as common a problem as antibiotic resistance.

Another approach to controlling pathogenic bacteria has been proposed, which relies on using molecular biological techniques to prevent the expression of antibiotic resistance genes in pathogenic bacteria (U.S. Pat. No. 5,976,864, incorporated herein by reference in its entirety). In this method, a nucleic acid construct encoding an “external guide sequence” specific for the targeted antibiotic resistance gene is introduced into the pathogenic bacterial cells. The sequence is expressed, hybridizes with messenger RNA (mRNA) encoding the antibiotic resistance gene product, and renders such mRNA sensitive to cleavage by the enzyme RNAse P. Such a system also has limited utility, since it targets specific antibiotic resistance genes. While the system may be effective in overcoming resistance based on expression of those specific genes, continued use of the antibiotics places selective pressure on the bacteria to mutate other genes and develop resistance to the antibiotic by another mechanism.

It is clear from the foregoing discussion that current alternatives to antibiotic use are limited and suffer many of the same drawbacks as antibiotic use itself. A method of controlling unwanted traits in bacteria that is flexible in range and that cannot be overcome by the bacteria by a single or small number of mutations is desirable in the art.

### BRIEF SUMMARY OF THE INVENTION

In one aspect, the present invention relates to a conjugative displacing plasmid for displacing a harmful plasmid in target bacteria. The conjugative displacing plasmid contains an origin of replication, an origin of conjugative transfer and an element that can inhibit the replication of a harmful plasmid in target bacteria. The element is genetically engineered into the conjugative displacing plasmid at a location outside the origin of replication. Generally speaking, any element that can inhibit the replication of a harmful plasmid, which makes the conjugative displacing plasmid and the harmful plasmid incompatible, can be engineered into the conjugative displacing plasmid. In one embodiment, the element is an iteron sequence which a harmful plasmid relies on for replication.

In another aspect, the present invention relates to a donor cell that contains the conjugative displacing plasmid described above.

In still another aspect, the present invention relates to a method of displacing a harmful plasmid in a target bacterial population. The method involves conjugating a donor cell that contains the conjugative displacing plasmid described above to a recipient bacterial cell such that the conjugative displacing plasmid is transferred from the donor cell to the recipient cell. When the recipient bacterial cell replicates, the harmful plasmid is lost from the growing bacterial population.

### BRIEF DESCRIPTION OF THE SEVERAL VIEWS OF THE DRAWINGS

Not applicable.

### DETAILED DESCRIPTION OF THE INVENTION

The present invention provides a novel anti-virulence strategy in which a harmful plasmid in target bacterial cells is displaced by a non-harmful plasmid without killing the bacterial cells. For the purpose of this invention, “displacing a harmful plasmid” means reducing the number of the harmful plasmid in or evicting the harmful plasmid completely from a target bacterial population. A harmful plasmid is defined herein as a plasmid that confers an unwanted trait to a host bacterial cell. A non-harmful plasmid is defined herein as a plasmid that does not confer any unwanted trait to a either a donor or a recipient bacterial cell. The employment of the novel strategy of the present invention can convert target bacterial cells with an unwanted trait to cells without the unwanted trait. Examples of unwanted traits include but are not limited to antibiotic-resistance and virulence to plants and human and nonhuman animals.

There are many situations in which eliminating an unwanted trait from target bacteria rather than killing these bacteria is desirable. For example, in growth-limiting situations such as the case of bio-films, simply killing one or more members of a bacterial community will generate a void into which co-resident species of bacteria including those that carry a harmful plasmid can grow and expand. In other situations, a change in composition and dynamics of a bacterial community caused by selectively killing certain bacteria may lead to undesirable consequences. In another example, pathogenic bacteria with the harmful trait eliminated will persist long enough for a human or non-human animal to develop a normal immune response producing antibodies for prevention of future infections.

The strategy of the present invention involves genetically engineering into a non-harmful plasmid an incompatibility element, which is defined herein as an element that can inhibit the replication of a harmful plasmid in a target bacterium and hence make the non-harmful plasmid and the harmful plasmid incompatible. For example, if a harmful plasmid relies on an iteron sequence to replicate, one or more copies of the iteron sequence can be genetically engineered into a non-harmful plasmid. When the non-harmful plasmid is introduced into a bacterial cell that contains the harmful plasmid, it binds to replication proteins that would have otherwise bound to the iteron sequence on the harmful plasmid. Thus, the replication of the harmful plasmid is inhibited leading to its ultimate eviction from the target bacteria. Other means of inhibiting the replication of a specific plasmid that rely on the general property of incompatibility can also be genetically engineered into a non-harmful plasmid for inhibiting the replication of a harmful plasmid in target bacteria (R. P. Novick “Plasmid incompatibility” Microbiol. Rev. 1987 p. 381-395, incorporated herein by reference in its entirety).

The strategy of the present invention also utilizes the highly efficient conjugation system to transfer a non-harmful plasmid from a donor bacterial cell to a target (recipient) bacterial cell. When both an incompatibility element and an origin of conjugative transfer along with an origin of replication are genetically engineered into a non-harmful plasmid, the non-harmful plasmid is then also termed as a conjugative displacing plasmid indicating that the plasmid can be transferred to a target cell through conjugation and can displace a harmful plasmid in the target cell.

The present invention is described below using iteron sequences as an example. However, other components of a harmful plasmid's replication machinery that can cause plasmid incompatibility with another plasmid can also be taken advantage of for the design of an incompatibility element. A skilled artisan is familiar with those components and can readily replace the iteron sequence with another incompatibility element (may involve small RNA molecules and proteins) to practice the present invention.

Iterons are iterated DNA sequences present in origins (start sites for initiation of DNA replication) of many bacterial plasmids; their presence is essential for replication, yet they can inhibit replication of the parental plasmid when they are cloned into another plasmid (reviewed by Helinski, D. R., A. E. Toukdarian, and R. P. Novick (1996) “Replication control and other stable maintenance mechanisms of plasmids,” p. 2295-2324 in F. Neidhardt, J. L. Ingraham, E. C. C. Lin, K. Brooks Low, B. Magasanik, W. Reznikoff, M. Riley, M. Schaechter and H. E. Umbarger (ed.), Escherichia coli and Salmonella Cellular and Molecular Biology, 2^{nd} ed, vol. 2 ASM Press, Washington D.C.; and Kim, K., and R. J. Meyer “Copy number of the broad-host range plasmid R1162 is determined by the amounts of essential plasmid-encoded proteins,” J. Mol. Biol., 185: 755-767, 1985, both of which are herein incorporated by reference as if set forth in their entirety).

In one aspect, the present invention relates to a conjugative displacing plasmid that contains an origin of replication (e.g., oriV), an origin of conjugative transfer (e.g., oriT), one or more copies of an iteron sequence that is used by a harmful plasmid for replication, and optionally, a screenable (selective marker). The iteron sequence(s) on the conjugative displacing plasmid is/are located outside the origin of replication. The copy number on the conjugative displacing plasmid is sufficient to inhibit the replication of the harmful plasmid. Preferably, the conjugative displacing plasmid contains 3 to 20 copies, and most preferably 5 to 10 copies of an iteron sequence. One of ordinary skill in the art will appreciate that more than one type of iteron sequences can be engineered into a single conjugative displacing plasmid so that the plasmid may be used for displacing more than one type of harmful plasmids. If it were not for the iteron sequences and any other incompatibility element that is genetically engineered into the conjugative displacing plasmid, the conjugative displacing plasmid would have been otherwise compatible with its target harmful plasmid. A skilled artisan can readily determine the iteron sequence of a harmful plasmid and then construct a conjugative displacing plasmid accordingly.

If it is desirable to make a conjugative displacing plasmid self-transmissible, genes that are necessary for the conjugative transfer (e.g., tra genes) of the plasmid from a donor cell to a recipient cell are also genetically engineered into the plasmid. Once such a conjugative displacing plasmid is transmitted from an original donor cell to a first recipient cell, it is capable of transmitting itself again to subsequent recipients. A plasmid of this type is more effective in displacing harmful plasmids than the non-self-transmissible type.

For a non-self-transmissible conjugative displacing plasmid, no or at least not all of the tra genes necessary for the conjugative transfer are present in the plasmid. A helper plasmid and/or a host cell contain either all or the rest of the tra genes. When a helper plasmid is used, it also contains an origin of replication and optionally a screenable (selective) marker. The non-self-transmissible conjugative displacing plasmid allows conjugative transfer of the conjugative displacing plasmid, but not or not all of the tra genes. Since the conjugative displacing plasmid lacks at least some of the tra genes necessary to convert a recipient cell into a potential donor cell, the conjugation can be controlled to occur with one-to-one stoichiometry. Typically, the recipient cell will not transfer the conjugative displacing plasmid further to a second recipient.

Depending on the harmful plasmid to be evicted, suitable plasmids that can be used to construct a conjugative displacing plasmid of the present invention include but are not limited to antibiotic-resistance-conferring plasmids R6K, RK2, pCU1, pSa, pCTTI, pCI305, pFA3, PIP404 (reviewed in Helinski, D. R., A. E. Toukdarian, and R. P. Novick 1996, incorporated herein by reference as if set forth in its entirety), anthrax-conferring plasmids pXO1 and PXO2 (M. Mock and A. Fouet “Anthrax” in Annu. Rev. Microbiol., 55; 647-671, 2001), plaque-conferring plasmids (P. Hu, J. Ellioott, P. McCready, E. Skowronski, J. Games, A. Kobayashi, R. R. Brubaker and E. Garcia, “Structural organization of Virulence-associated Plasmids of Yersinia pestis,” J. Bacteriol. 180; 5192-5202, 1998; L. E. Lindler, G. V. Plano, V. Burland, G. F. Mayhey and F. Blattner, Infection and Immunity 66: 5731-5742, 1998), and fitness-conferring plasmid pEA29 of plant pathogen Erwinia amylovora (G. C. McGhee and A. L. Johnes, “Complete nucleotide sequence of ubiquitous plasmid pEA29 from Erwinia amylovora strain Ea88: Gene organization and intraspecies variation,” Applied and Enir. Microbiol. 66, p 4897-4907, 2000).

The selection of oriV for a conjugative displacing plasmid will affect its range of potential recipients. In most instances, it is preferable to target a specific recipient for the conjugative displacing plasmid. Such instances include, but are not limited to, using the conjugative displacing plasmid for displacing harmful plasmids in Enterobacteria, Enterococci, Staphylococci and non-sporulating Gram-positive pathogens such as Nocardia and Mycobacterium sp. Examples of selective host range plasmids from which such oriV's may be obtained include, but are not limited to, P1 and F.

In instances where it is desirable to affect a wide variety of recipients, a broad range oriV is employed. Examples of broad range (“promiscuous”) plasmids from which oriVs may be obtained include, but are not limited to, R6K and its derivatives, RK2 and its derivatives (i.e., RP4), p15A and its derivatives, RSF1010 and its derivatives, pMV158 and its derivatives and pPS10 and its derivatives (D. E. Rawlings and E. Tietze “Comparative Biology of IncQ and IncQ-like plasmids,” Microbiol. And Mol. Biol. Reviews 65, 481-496, 2001; Gloria del Solar, J. C. Alonso, M. Espinosa and R. Diaz-Orejas. Mol. Microbiol., 21, 661-666, 1996).

As used herein, the term “range” (or “host range”) refers generally to parameters of both the number and diversity of different bacterial species in which a particular plasmid (natural or recombinant) can replicate. Of these two parameters, one skilled in the art would consider diversity of organisms as generally more defining of host range. For instance, if a plasmid replicates in many species of one group, e.g., Enterobacteriaceae, it may be considered to be of narrow host range. By comparison, if a plasmid is reported to replicate in only a few species, but those species are from phylogenetically diverse groups, that plasmid may be considered of broad host range. As discussed above, both types of plasmids will find utility in the present invention.

Conjugative transfer (tra) genes have been characterized in many conjugative bacterial plasmids. The interchangeability between the gene modules conferring the ranges of hosts susceptible for conjugal transfer and vegetative replication include Gram-positive and Gram-negative species. Examples of characterized tra genes that are suitable for use in the present invention include, but are not limited to, the tra genes from: (1) F (Firth, N., Ippen-Ihler, K. and Skurray, R. A. 1996, Structure and function of F factor and mechanism of conjugation. In: Escherichia coli and Salmonella, Neidhard et al., eds., ASM Press, Washington D.C.); (2) R6K (Nunez et al., Mol. Microbiol. 24: 1157-1168, 1997); and (3) Ti (Ferrand et al., J. Bacteriol. 178: 4233-4247, 1996). Additional tra genes that find use with the present invention include, but are not limited to, those described in U.S. Pat. Nos. 6,180,406 and 6,251,674, both of which are herein incorporated by reference in their entirety.

In preferred embodiments of the present invention, a conjugative displacing plasmid or a helper plasmid contains a screenable (selective) marker gene. In traditional molecular biological manipulations of recombinant bacteria, a screenable marker gene is often an antibiotic resistance gene. Since the present invention is designed to avoid further spread of antibiotic resistance, an alternative screenable marker system is preferred for use in the present invention. Accordingly, although antibiotic resistance markers can be used in laboratory tests, preferred selectable markers include, but are limited to, nutritional markers, i.e., any auxotrophic strain (e.g., Trp⁻, Leu⁻, Pro⁻) containing a plasmid that carries a complementing gene (e.g., trp⁺, leu⁺, pro⁺).

In another aspect, the present invention is a donor cell that contains a conjugative displacing plasmid of the present invention. The donor cell also contains, whether on the conjugative displacing plasmid, another plasmid (e.g., a helper plasmid) or the bacterial genome, genes that are necessary for conjugative transfer of the conjugative displacing plasmid. A skilled artisan knows how to introduce a conjugative displacing plasmid into a donor cell. Depending on the target bacterial cell, a skilled artisan can readily determine bacterial cells that are suitable as donor cells and make such donor cells having the characteristics described above. A skilled artisan will appreciate that a single donor bacterial strain might harbor multiple conjugative displacing plasmids designed for one or more types of harmful plasmids that need to be displaced.

In preferred embodiments, an environmentally safe donor strain is used for the above-described conjugative displacing plasmids. For example, a donor strain can be any one of the many non-pathogenic bacterial strains associated with the body of human and non-human animals and plants. Preferably, non-pathogenic bacteria that colonize the non-sterile parts of the body (e.g., skin, digestive tract, urogenital region, mouth, nasal passages, throat and upper airway, ears and eyes) are utilized as donor cells. Examples of particularly preferred donor bacterial species include, but are not limited to: (1) non-pathogenic strains of Escherichia coli (E. coli F 18 and E. coli strain Nissle 1917), (2) various species of Lactobacillus (such as L. casei, L. plantarum, L. paracasei, L. acidophilus, L. fermentum, L. zeae and L. gasseri), (3) other nonpathogenic or probiotic skin- or GI-colonizing bacteria such as Lactococcus, Bifidobacteria, Eubacteria, Erwinia, Xanthomonas pseudomonas, and (4) bacterial mini-cells, which are anucleoid cells destined to die but still capable of transferring plasmids (see; e.g., Adler et al., Proc., Nat., Acad., Sci. USA 57: 321-326, 1970; Frazer and Curtiss III, Current Topics in Microbiology and Immunology 69: 1-84, 1975; U.S. Pat. No. 4,968,619 to Curtiss III, incorporated by reference herein in its entirety).

It should be noted that in addition to bacterial cells described above, donor cells can also include non-dividing cells such as temperature-sensitive mutants, chromosome-less mini-cells and maxi-cells, all of which are described later in the specification.

In still another aspect, the present invention is a method of reducing the number of or eliminating completely a harmful plasmid in a target bacterial population by displacing the harmful plasmid with a nonharmful plasmid. The method involves bringing a donor bacterial cell of the present invention into conjugative proximity to a target bacterial cell such that the donor bacterial cell conjugates with the target bacterial cell resulting in the transfer of conjugative displacing plasmid from the donor cell into the target cell. The conjugative displacing plasmid then inhibits the replication of the harmful plasmid in the target cell and eventually causes the loss of the harmful plasmid from the growing target bacterial population.

The method of the present invention for displacing a harmful plasmid in a target bacterial population finds utility in a variety of human, veterinary, agronomic, horticultural and food processing settings. A skilled artisan can readily formulate a composition containing the proper donor bacterial cells for a particular application. A skilled artisan can also formulate the composition for a specific route of administration.

For human and veterinary use, and depending on the cell population or tissue targeted for protection, the following modes of administration of the bacteria of the invention are contemplated: topical, oral, nasal, pulmonary/bronchial (e.g., via an inhaler), ophthalmic, aural, rectal, urogenital, subcutaneous, intraperitoneal and intravenous. The bacteria preferably are supplied as a pharmaceutical preparation, in a delivery vehicle suitable for the mode of administration selected for the human or nonhuman animal being treated.

For instance, to deliver the bacteria to the gastrointestinal tract or to the nasal passages, the preferred mode of administration is by oral ingestion or nasal aerosol, or by feeding (alone or incorporated into the subject's feed or food). In this regard, it should be noted that probiotic bacteria, such as Lactobacillus acidophilus, are sold as gel capsules containing a lyophilized mixture of bacterial cells and a solid support such as mannitol. When the gel capsule is ingested with liquid, the lyophilized cells are re-hydrated and become viable, colonogenic bacteria. Thus, in a similar fashion, donor bacterial cells of the present invention can be supplied as a powdered, lyophilized preparation in a gel capsule, or in bulk for sprinkling into food or beverages. The re-hydrated, viable or non-viable bacterial cells will then populate and/or colonize sites throughout the upper and lower gastrointestinal system, and thereafter come into contact with the target pathogenic bacteria.

For topical applications, the bacteria may be formulated as an ointment or cream to be spread on the affected skin or mucosal surface. Ointment or cream formulations are also suitable for rectal or vaginal delivery, along with other standard formulations, such as suppositories. The appropriate formulations for topical, vaginal or rectal administration are well known to medicinal chemists.

Other uses for the donor bacteria of the invention are also contemplated. These include a variety of agricultural, horticultural, environmental and food processing applications. In such applications, formulation of donor bacteria as solutions, aerosols, or gel capsules are contemplated. For example, in agriculture and horticulture, various plant pathogenic bacteria may be targeted in order to minimize plant disease. Donor cells of conjugative displacing plasmids can be applied. Food and plant surfaces can be targeted as well. Donor cells of conjugative displacing plasmid can be applied to meat and other food, including animal feed, to displace harmful plasmids in bacteria associated with the food material. One example of a plant pathogen suitable for targeting is Erwinia amylovora, the causal agent of fire blight which is known to harbor an iteron-containing plasmid pEA29 (McGhee and Johnes, 2000). Donor bacteria such as comensal Erwinia herbicola can be adopted as the delivery systems of conjugative displacing plasmids. Like chemical antibiotics, the donor bacteria can be aerosolized or delivered to infected flowers (stigmas) using honey bees as vectors (S. V., Thomson, D. R. Hansen, K. M. Flint and J. D. Vandenberg “Dissemination of bacteria antagonistic to Erwinia amylovora by honey bees,” Plant Disease 76, 1052-1056, 1992). Similar strategies may be utilized to reduce or prevent wilting of cut flowers and vegetables.

In preferred embodiments of the present invention, certain features are employed in the plasmids and donor cells of the invention to minimize potential risks associated with the use of DNA or genetically modified organisms in the environment. For instance, in environmentally-sensitive circumstances, it is preferable to utilize non-self-transmissible plasmids. Instead, the plasmids will be mobilizable by host-coded conjugative machinery. As discussed hereinabove, this may be accomplished in some embodiments by integrating into the host chromosome all tra genes whose products are necessary for the assembly of conjugative machinery. In such embodiments, conjugative displacing plasmids are configured to possess only an origin of transfer (oriT). This feature prevents the recipient from transferring the conjugative displacing plasmid further.

Another biosafety feature comprises utilizing conjugation systems with pre- determined host-ranges. As discussed above, certain elements are known to function only in few related bacteria (narrow-host-range) and others are known to function in many unrelated bacteria (broad-host-range or promiscuous) (del Solar et al., Mol. Microbiol. 32: 661-666, 1996; Zatyka and Thomas, FEMS Microbiol. Rev. 21: 291-319, 1998). Also, many of those conjugation systems can function in either gram-positive or gram-negative bacteria but generally not in both (del Solar, 1996, supra; Zatyka and Thomas, 1998, supra).

Also as discussed in detail above, inadvertent proliferation of antibiotic resistance is minimized in this invention by avoiding the use of antibiotic resistance markers. In a preferred alternative approach, the gene responsible for the synthesis of an amino acid (i.e. serine) can be mutated, generating the requirement for this amino acid in the donor. Such mutant donor bacteria will prosper on media lacking serine provided that they contain a plasmid with the ser gene whose product is needed for growth. Thus, the invention contemplates the advantageous use of plasmids containing the ser gene or one of many other nutritional genetic markers. These markers will permit selection and maintenance of the conjugative displacing plasmids in donor cells.

Another biosafety approach comprises the use of restriction-modification systems to modulate the host range of conjugative displacing plasmids. Conjugation and plasmid establishment upon its conversion from a single-stranded DNA molecule to a double-stranded DNA molecule (Zatyka and Thomas, 1998, supra) are expected to occur more frequently between taxonomically related species in which plasmid can evade restriction systems and replicate. Type II restriction endonucleases make a double-strand break within or near a specific recognition sequence of duplex DNA. Cognate modification enzymes can methylate the same sequence and protect it from cleavage. Restriction-modification systems (RM) are ubiquitous in bacteria and archaebacteria but are absent in eukaryotes. Some of RM systems are plasmid-encoded, while others are on the bacterial chromosome (Roberts and Macelis, Nucl. Acids Res. 24: 223-235, 1998). Restriction enzymes cleave foreign DNA such as viral or plasmid DNA when this DNA has not been modified by the appropriate modification enzyme. In this way, cells are protected from invasion of foreign DNA. Thus, by using a donor strain producing one or more methylases, cleavage by one or more restriction enzymes could be evaded in the target bacteria. Another approach can employ site-directed mutagenesis to produce plasmid DNA that is either devoid of specific restriction sites or that comprises new sites, protecting or making plasmid DNA vulnerable (in pre-determined bacterial hosts), respectively, against endonucleases.

Preferred embodiments of the present invention also utilize environmentally safe bacteria as donors. For example, delivery of DNA vaccines by attenuated intracellular gram-positive and gram-negative bacteria has been reported. In addition, the donor strain can be one of the many harmless bacterial strains that colonize the non-sterile parts of the body (e.g., skin, gastrointestinal, urogenital, mouth, nasal passages, throat and upper airway systems). Examples of preferred donor bacterial species are set forth hereinabove.

In another strategy, non-dividing, non-growing donors are utilized instead of living cells. As discussed above, mini-cells and maxi-cells are well studied model systems of metabolically active but nonviable bacterial cells. Mini-cells lack chromosomal DNA and are generated by special mutant cells that undergo asymmetric cell division which leads to one progeny cell with two copies of chromosome and another “cell” (mini-cell) which is chromosome-less. If the cell contains a multicopy plasmid, many of the mini-cells will contain plasmids. Mini-cells are not viable since they neither divide nor grow. However, mini-cells that possess conjugative plasmids are capable of conjugal replication and transfer of plasmid DNA to living recipient cells. (Adler et al., 1970, supra; Frazer and Curtiss, 1975, supra; U.S. Pat. No. 4,968,619, supra).

Maxi-cells can be obtained from a strain of E. coli that carries mutations in the key DNA repair pathways (recA, uvrA and phr). Because maxi-cells lack so many DNA repair functions, they die upon exposure to low doses of UV. Importantly, plasmid molecules (e.g., pBR322) that do not receive an UV hit continue to replicate. Transcription and translation (plasmid-directed) can occur efficiently under such conditions (Sancar et al., J. Bacteriol. 137: 692-693, 1979), and the proteins made prior to irradiation should be sufficient to sustain conjugation. This is supported by the following three observations: i) streptomycin-killed cells remain active donors; ii) transfer of conjugative plasmids can occur in the presence of antibiotics that prevent de novo gene expression (Heineman and Ankenbauer, 1993, J. Bacteriol. 175, 583-588; Cooper and Heineman, 2000. Plasmid 43, 171-175); and iii) maxi-cells can transfer plasmid DNA to live recipients. It should also be noted that the conservation of recA and uvrA genes among bacteria should allow maxicells of donor strains other than E. coli to be obtained.

Also contemplated for use in the invention are any of the modified microorganisms that cannot function because they contain temperature-sensitive mutation(s) in genes that encode for essential cellular functions (e.g., cell wall, protein synthesis, RNA synthesis, as described, for example, in U.S. Pat. No. 4,968,619, supra). A conjugative displacing plasmid can also contain a temperature-sensitive mutation in a replication-related gene so that it can replicate only at temperatures below 37° C. Hence, its replication will occur in bacteria applied on skin but it will not occur if such bacteria break into the body's core.

The invention will be more fully understood upon consideration of the following non-limiting examples.

### EXAMPLE

Methods

Conjugation was performed according to the following protocol: Cultures of donors and recipients were grown overnight in 5 mL of Luria Broth supplemented with appropriate antibiotics. A number of viable cells in each culture was determined by plating dilutions on LB media. Conjugation was carried out on nitrocellulose filters for 2 hours at 37° C. in the absence of selection. The cells or their combinations were removed from filters by vortexing and dilutions were plated on the selective media. Plates were incubated at 37° C. overnight and colonies were counted.

Plasmids: DNA sequences of some plasmids constructed in Dr. Marcin Filutowicz's laboratory and used in this work are shown in the sequence listing: pUC9 (SEQ ID NO:1), pFL601 (SEQ ID NO:2), pFL604 (SEQ ID NO:3), pFL606 (SEQ ID NO:4) and pJWW204 (SEQ ID NO:5). The number of iterons in plasmids pFL602, pFL603 and pFL605 is inferred from restriction digests.

Donor strain: We used a donor strain E. coli S17.1 in which all tra genes that are needed for conjugation were integrated into the chromosome. The strain can mobilize a broad range of the oriT-containing plasmids (R. Simon, U. Priefer and A. Puhler. “A broad host range mobilization system for in vitro genetic engineering: Transposon mutagenesis in Gram-negative bacteria,” Biotechnology 784-791, 1983).

Mobilizable conjugative displacing plasmids: Plasmids used in this study contained one iteron (pFL602), two iterons (pFL603), three iterons (pFL604), four iterons (pFL605) or seven iterons (pFL606). Plasmid pUC9 that does not contain any iteron sequence was used as a control. These plasmids also contained a penicillin resistance gene (bla).

Recipient strain: The recipient strain in conjugation experiments was RLG315, which was a Rifampicin-resistant derivative of W1110 strain into which a tester plasmid (pJWW204, an R6K derivative) was introduced by transformation.

Analysis of transfer efficiency and ability to displace R6K derivatives: The conjugative displacing plasmids were tested for plasmid transfer efficiency as well as their ability to displace an R6K derivative (pJWW204). Mating pairs of the donor strain containing the displacing plasmid and the recipient strain were established without any antibiotic selection for 2 hours. Mixtures of bacteria (donor, recipient and mixtures of both) were then plated out on plates supplemented with rifampicin alone, penicillin alone, chloramphenicol alone, and combinations of those antibiotics; only the trans-conjugants were expected to grow in the presence of rifampicin, chloramphenicol (recipient marker) and penicillin (donor marker). The plates were observed for colony growth.

Results

The plasmid transfer efficiency was nearly 100% during 2 hours of filter-mating. A decrease in the number of trans-conjugants was observed with an increase in the number of iterons contained in the conjugative displacing plasmid. No colonies were observed in matings with plasmids containing either five or seven iterons when the selection was employed for chloramphenicol, rifampicin and penicillin. When conjugative mixtures were plated on penicillin- and rifampicin-containing media and then screened by replica-plating for resistance to chloramphenicol (encoded by pJWW204), all colonies that received a conjugative donor plasmid lacking iterons (pUC9) mainained chloramphenicol-resistance phenotype. This was expected because the donor plasmid and pJWW204 are members of different incompatibility groups and so could be maintained in the same bacterial cell. In contrast, clones that contained iterons had a reduced (pFL602, pFL603, pFL604 and pFL605) or undetectable level of chloramphenicol resistance (pFL606). The frequency with which chloramphenicol-resistance occurred was indicative of the effectiveness of the iteron-containing plasmids to displace pJWW204 plasmid from the recipient cells.

All publications and patents mentioned in the above specification are herein incorporated by reference as if expressly set forth herein. Although the invention has been described in connection with specific embodiments, it is understood that the invention is not limited to such specific embodiments but encompasses all such modifications and variations apparent to a skilled artisan that fall within the scope of the appended claims.

We claim:

1. A non-harmful conjugative displacing plasmid for displacing a harmful plasmid comprising:
a) an origin of replication for synthesizing the non-harmful conjugative displacing plasmid in a bacterial cell;
b) an element that can inhibit the replication of the harmful plasmid located outside the origin of replication; and
c) an origin of transfer from which conjugative transfer of the non-harmful conjugative displacing plasmid initiates from a donor bacterial cell to at least one recipient bacterial cell.

2. The non-harmful conjugative displacing plasmid of claim 1 wherein the element that can inhibit the replication of the harmful plasmid is an iteron used by the harmful plasmid for replication.

3. The non-harmful conjugative displacing plasmid of claim 2 further comprising:
at least one screenable marker gene.

4. The non-harmful conjugative displacing plasmid of claim 2 further comprising:
at least one transfer gene for transferring the non-harmful conjugative displacing plasmid from a donor bacterial cell to a recipient bacterial cell.

5. The non-harmful conjugative displacing plasmid of claim 2, wherein the non-harmful conjugative displacing plasmid is R6K containing at least three copies of R6K iteron outside the origin of replication.

6. A donor cell comprising:
a non-harmful conjugative displacing plasmid of claim 1;
all transfer genes necessary for conferring upon the donor cell the ability to conjugatively transfer the non-harmful conjugative displacing plasmid from the donor cell to a recipient bacterial cell.

7. The donor cell of claim 6, wherein at least some of the transfer genes are carried by a helper plasmid within the donor cell or by the donor cell genome, such that the non-harmful conjugative displacing plasmid is transmissible from the donor cell to a recipient bacterial cell, but is not further self-transmissible from the recipient cell to another recipient cell.

8. The donor cell of claim 6, wherein all of the transfer genes are located on the non-harmful conjugative displacing plasmid, such that the non-harmful conjugative displacing plasmid is self-transmissible from the donor cell to a recipient bacterial cell, and further from the recipient cell to another recipient cell.

9. The donor cell of claim 6, wherein the donor cell is a non-pathogenic strain of bacteria selected from the group consisting of Escherichia coli, Lactobacillus spp., Lactococcus, Bifidobacteria, Eubacteria, and bacterial minicells.

10. The donor cell of claim 6, wherein the recipient bacterial cell is a pathogenic strain of bacterium selected from the group consisting of Campylobacter spp., Enterobacter spp., Enterococcus spp., Escherichia coli, Gardnerella vaginalis, Haemophilus spp., Helicobacter pylorii, Mycobacterium tuberculosis, Propionobacter acnes, Pseudomonas aeruginosa and other Pseudomonas spp., Salmonella typhimurium, Shigella spp. and Staphylococcus spp.

11. The donor cell of claim 6, wherein the donor bacterial cell is a bacterium of strain S17.1.

12. A pharmaceutical preparation for displacing a harmful plasmid in a target bacterial population in a subject, the preparation comprising the donor cell of claim 6 formulated for a pre-determined route of administration to the subject.

13. The pharmaceutical preparation of claim 12, wherein the pre-determined route of administration is selected from the group consisting of a topical route, an oral route, a nasal route, a pulmonary route, an ophthalmic route, an aural route, a rectal route, a urogenital route, a subcutaneous route, an intraperitoneal route and an intravenous route.

14. A method for displacing a harmful plasmid with a non-harmful plasmid in a bacterial population, the method comprising the steps of:
providing a donor cell according to claim 6; and
conjugating the donor cell to a recipient bacterial cell such that a non-harmful conjugative displacing plasmid is transferred from the donor cell to the recipient cell,
wherein the nonharmful conjugative displacing plasmid inhibits replication of the harmful plasmid in the recipient cell and when the recipient bacterial cell replicates, the harmful plasmid is lost in the growing bacterial population.

15. The method of claim 14 wherein replication of the harmful plasmid involves an iteron sequence and the donor cell contains a conjugative displacing plasmid that contains the same iteron sequence outside the origin of replication.

16. The method of claim 14, wherein the non-harmful conjugative displacing plasmid is R6K carrying 3 copies of iteron of R6K.

17. The method of claim 14, wherein the recipient bacterium resides in a subject selected from a human or non-human animal, a plant, or a food source.


SEQUENCE LISTING (5 sequences)

SEQ ID NO: 1 - DNA, 2665 bp, Artificial
  genetically engineered plasmid
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggctgcaggtcgacggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 2 - DNA, 3450 bp, Artificial
  genetically engineered plasmid
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggctgcaggtcgacggatcacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgacccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 3 - DNA, 3567 bp, Artificial
  genetically engineered plasmid
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggctgcagtgaattcccggggatccgtctaattttattgttcaaacatgagagcttagtacgtgaaacatgagagcttagtacgttagccatgagagcttagtacgtgacctgcagccaagcttggtcgacggatcacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgacccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 4 - DNA, 3615 bp, Artificial
  genetically engineered plasmid
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggctgcagtaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtcgacggatcacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgacccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 5 - DNA, 3267 bp, Artificial
  genetically engineered plasmid
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgccattcgccattcaggctgcgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagggccagtgaattcagtgtcagccgttaagtgttcctgtgtcactgaaaattgctttgagaggctctaagggcttctcagtgcgttacttccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgctgatcttcagatccacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgtcgacctgcagccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg